(19) United States
(12) Patent Application Publication
(10) Pub. No.: US 2025/0242173 A1
(43) Pub. Date: Jul. 31, 2025

(54) MULTI-WAVELENGTH LASER TREATMENT DEVICE AND USAGE METHOD FOR TRICHOPHYTON RUBRUM INDUCED ONYCHOMYCOSIS

(71) Applicant: Air Force Medical Center, PLA, Beijing (CN)

(72) Inventor: Hong CAI, Beijing (CN)

(21) Appl. No.: 18/903,560

(22) Filed: Oct. 1, 2024

(30) Foreign Application Priority Data
Jan. 31, 2024 (CN) 202410131328.0

(51) Int. Cl. A61N 5/067; A61N 5/06

(52) U.S. Cl. CPC A61N 5/067; A61N 5/0616; A61N 5/0624; A61N 2005/0626; A61N 2005/063; A61N 2005/0632; A61N 2005/0644; A61N 2005/0659; A61N 2005/0663; A61N 2005/0666

(57) ABSTRACT

The multi-wavelength laser treatment device comprises: a drive control circuit, a power supply, an optical system, an air-cooled heat sink, an output fiber optic, and a hand tool. The hand tool includes a moving piece, a convex lens, and a bracket. The bracket serves as a light emitting end face. The optical system includes a first laser chip, a second laser chip, a third laser chip, a first optical mirror, a second optical mirror, and a coupler. The light beam is coupled to the output fiber optic through the coupler and emits light from the light emitting end face of the hand tool. Wherein, the first laser chip, the second laser chip and the third laser chip have a light emitting wavelength of 405 nm, 1064 nm or 632 nm. The moving piece is used to fix the light emitting and can switch between a converging position and a diverging position.

### CROSS-REFERENCE TO RELATED APPLICATION

This application claims priority from the Chinese patent application 202410131328.0 filed Jan. 31, 2024, the content of which is incorporated herein in the entirety by reference.

### TECHNICAL FIELD

The present disclosure relates to a multi-wavelength laser treatment device for trichophyton rubrum induced onychomycosis, and at the same time also relates to a method of using the multi-wavelength laser treatment device, belonging to the technical field of medical devices.

### BACKGROUND

Trichophyton rubrum is a human borne dermatophyte that often infects the skin and nails of humans, causing common superficial skin fungal diseases. It is the main pathogen for human onychomycosis.

At present, the principle of selective photothermal action is often used in clinical practice, and high energy density long pulse 1064 nm infrared laser is used as an important treatment method for trichophyton rubrum induced onychomycosis.

Currently, art known by the inventor(s) 1064 nm long pulse laser is generated by pulsed xenon lamp pumped Nd:YAG solid-state laser crystals. the Nd:YAG solid-state laser equipment requires a pump reflection cavity, laser resonant cavity lens group, fiber coupling device, etc., so the system size is large, and the debugging is relatively complex. Among them, the sensitive laser resonant cavity lens group needs to remain strictly parallel, so it is rather sensitive to shock vibration and transportation, and is not suitable for field operations.

In addition, due to the limitations of the environment and hygiene conditions in the wild and campsites, it is often impossible to ensure complete cleaning of the skin surface in real time, and the body surface often carries a large amount of pathogens such as bacteria and viruses. Therefore, the disinfection and sterilization of bacteria and viruses before laser treatment and the accelerated recovery after treatment are also quite necessary. That is, a portable laser treatment device integrating disinfection and sterilization, treatment and rehabilitation is needed for the treatment of trichophyton rubrum induced onychomycosis.

### SUMMARY

The primary technical problem to be solved by the present disclosure is to provide a multi-wavelength laser treatment device for trichophyton rubrum induced onychomycosis.

Another technical problem to be solved by the present disclosure is to provide a method of using the above-mentioned multi-wavelength laser treatment device.

To achieve the above technical objectives, the present disclosure adopts the following technical solutions:

According to a first aspect of the embodiments of the present disclosure, a multi-wavelength laser treatment device for trichophyton rubrum induced onychomycosis is provided, comprising a drive control circuit, a power supply, an optical system, an air-cooled heat sink, an output fiber optic, and a hand tool,
the hand tool includes a moving piece, a convex lens, and a bracket, the bracket serves as a light emitting end face for fitting to the skin,
the drive control circuit is powered by the power supply and controls the optical system, air-cooled heat sink, moving piece, and convex lens,
the optical system includes a first laser chip, a second laser chip, a third laser chip, a first optical mirror, a second optical mirror, and a coupler,
the light beam is coupled to the output fiber optic through the coupler and emits light from the light emitting end face of the hand tool;
wherein, the first laser chip, the second laser chip and the third laser chip have a light emitting wavelength of 405 nm, 1064 nm or 632 nm respectively, for emitting light beams of 405 nm, 1064 nm and 632 nm wavelengths in turn, the 405 nm is used for disinfection and sterilization of bacteria attached to the surface of and around the onychomycosis, the 1064 nm is used to treat the trichophyton rubrum induced onychomycosis, and the light beam of 632 nm wavelength is used for rehabilitation;
the moving piece is used to fix the light emitting end of the output fiber optic and can switch between a converging position and a diverging position. When the moving piece is in the converging position, the distance L from the light emitting end of the output fiber optic to the optical center of the convex lens satisfies: f<L<2f; when the moving piece is in the diverging position, the distance L from the light emitting end of the output fiber optic to the optical center of the convex lens satisfies: L<f.

Preferably, the second laser chip and the third laser chip are both located between the first laser chip and the coupler,
the light emitting direction of the first laser chip passes through the optical center of the coupler and is aligned with the centerline of the output fiber optic,
the light emitting direction of the second laser chip and the third laser chip is perpendicular to the light emitting direction of the first laser chip, and the third laser chip is closer to the coupler compared with the second laser chip.

Preferably, the optical center of the first optical mirror is located in the light emitting direction of the first laser chip, and the first optical mirror is tilted relative to the light emitting direction of the first laser chip.

Preferably, the incident surface of the first optical mirror towards the first laser chip is coated with an antireflective film to transmit the emitted light of the first laser chip;
the exit surface of the first optical mirror towards the coupler is coated with a reflective film to reflect the emitted light of the second laser chip.

Preferably, the optical center of the second optical mirror is located in the light emitting direction of the first laser chip, and the second optical mirror is tilted relative to the light emitting direction of the first laser chip.

Preferably, the incident surface of the second optical mirror towards the first laser chip is coated with an antireflective film to transmit the light beam from the first laser chip and the light beam from the second laser chip;
the exit surface of the second optical mirror towards the coupler is coated with a reflective film to reflect the light beam from the third laser chip.

According to a second aspect of the embodiments of the present disclosure, a method of using a multi-wavelength laser treatment device is provided, comprising the following steps to:
S1: Use a drive control circuit to move the light emitting end of an output fiber optic to a diverging position;
S2: Use the drive control circuit to power on one of a first laser chip, second laser chip, or third laser chip to emit a light beam of 405 nm wavelength, couple it to the output fiber optic, then diverge into a large spot through a convex lens for output;
S3: After a first preset time, stop the light beam of 405 nm wavelength and move the light emitting end of the output fiber optic to a converging position;
S4: Use the drive control circuit to power on one of the first laser chip, second laser chip, or third laser chip to emit a light beam of 1064 nm wavelength, couple it to the output fiber optic, then converge into a small spot through the convex lens for output;
S5: After a second preset time, stop the light beam of 1064 nm wavelength and move the light emitting end of the output fiber optic to a diverging position;
S6: Use the drive control circuit to power on one of the first laser chip, second laser chip, or third laser chip to emit a light beam of 632 nm wavelength, couple it to the output fiber optic, then diverge into a large spot through the convex lens for output, the diameter of the large spot being larger than that of the small spot;
S7: After a third preset time, stop the light beam of 632 nm wavelength and end the process.

Preferably, the current provided by the drive control circuit to the first laser chip is greater than the current provided to the second and third laser chips.

Preferably, the first laser chip is used to provide a light beam of 1064 nm wavelength; the second laser chip is used to provide a light beam of 405 nm wavelength; the third laser chip is used to provide a light beam 632 nm wavelength.

Preferably, the first preset time is 10˜60 seconds, and the third preset time is 10˜30 minutes.

Compared with the existing art, the invention conducts disinfection and sterilization, treatment and rehabilitation for trichophyton rubrum induced onychomycosis through three wavelengths, and a one-off operation is enough to make patients recover from the trichophyton rubrum induced onychomycosis, thereby reducing the number of visits required of patients, being suitable for field and other application scenarios with poor medical conditions or requiring urgent treatments. Moreover, the present disclosure utilizes semiconductor laser chips to provide lasers of different wavelengths and powered by lithium batteries or charging circuits, which is conducive to achieving portable design. Therefore, the present disclosure solves the problem of difficult medical treatment for patients with trichophyton rubrum induced onychomycosis among field workers.

### BRIEF DESCRIPTION OF THE DRAWINGS

FIG. 1 is a schematic diagram of the overall structure of a multi-wavelength laser treatment device for trichophyton rubrum induced onychomycosis in the embodiments of the present disclosure;

FIG. 2A is a schematic diagram of the optical system in FIG. 1;

FIG. 2B is another schematic diagram of the optical system in FIG. 1;

FIG. 3A is a schematic diagram of the spot adjustment optical path of the multi-wavelength laser treatment device for trichophyton rubrum induced onychomycosis in a first embodiment of the present disclosure;

FIG. 3B is a schematic diagram of the spot adjustment optical path of the multi-wavelength laser treatment device for trichophyton rubrum induced onychomycosis in a second embodiment of the present disclosure.

### DETAILED DESCRIPTION OF THE INVENTION

Below is a detailed and specific explanation of the technical content of the present disclosure in conjunction with the accompanying drawings and specific embodiments.

The technical concept in the embodiments of the present disclosure is to use an optical beam combiner lens group to select one of three wavelengths at different times for disinfection, treatment, and rehabilitation, thus realizing a one-off operation that achieves the treatment and rehabilitation effect that usually requires two separate operations within a certain period of time. Therefore, it is particularly suitable for situations where medical services are not fully available like in the wild, also suitable for situations where separate operations cannot be carried out due to insufficient time (such as seeking medical treatment in a different location), and situations where treatment and rehabilitation need to be completed in a short period of time due to the conditions of the disease.

### First Embodiment

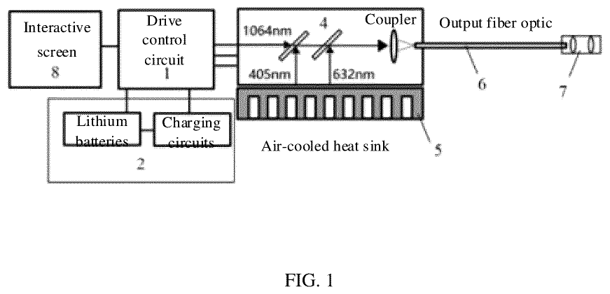

As shown in FIG. 1, the embodiments of the present disclosure provide a multi-wavelength laser treatment device 100 for trichophyton rubrum induced onychomycosis, comprising a drive control circuit 1, a power supply 2, an optical system 4, an air-cooled heat sink 5, an output fiber optic 6, and a hand tool 7. More preferably, it also comprises an interactive screen 8.

Drive control circuit 1 receives power from power supply 2, and controls optical system 4, air-cooled heat sink 5, hand tool 7, and interactive screen 8. Optical system 4 is used to select laser of a specific wavelength based on the signal from drive control circuit 1, couple it to output fiber optic 6, to provide laser of currently required wavelength to hand tool 7.

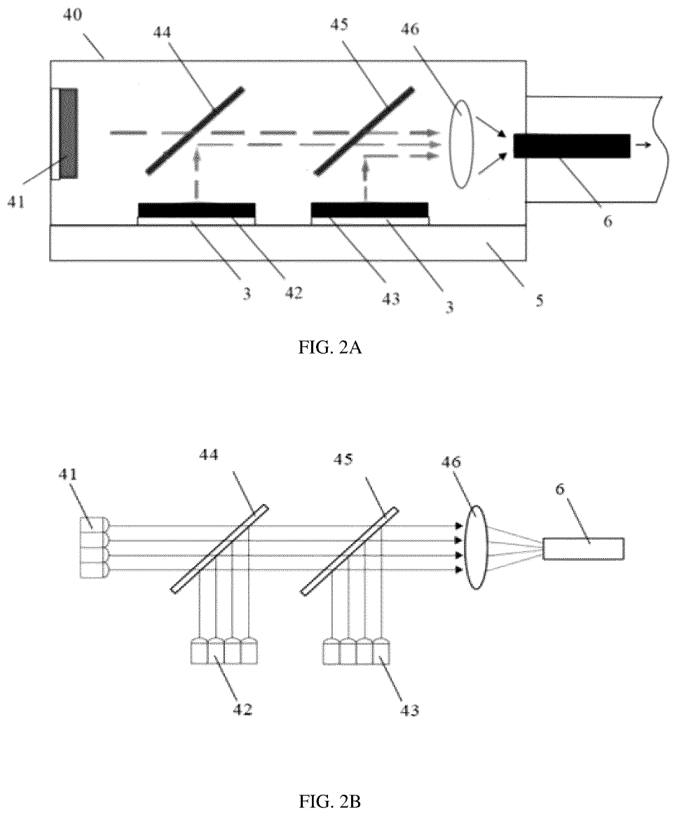

To be specific, as shown in FIG. 2A, optical system 4 includes a first laser chip 41, a second laser chip 42, a third laser chip 43, a first optical mirror 44, a second optical mirror 45, and a coupler 46.

In order to treat trichophyton rubrum induced onychomycosis, it is necessary to first use laser of 405 nm for disinfection and sterilization; then use laser of 1064 nm for treatment; and in the end, use laser of 632 nm for accelerated rehabilitation. Therefore, the emitting wavelengths of the first laser chip 41, second laser chip 42, and third laser chip 43 are one of 405 nm, 1064 nm or 632 nm respectively, for emitting light beams of 405 nm, 1064 nm and 632 nm wavelengths in turn. In this embodiment, the first laser chip 41 emits laser of 1064 nm; the second laser chip 4 emits laser of 405 nm; and the third laser chip 43 is a 632 nm laser.

The first laser chip 41 is a laser for treatment, which uses a high-power 1064 nm semiconductor laser with peak power of several hundred watts to replace the lamp pumped Nd:YAG laser in the existing art. It can increase the electro-optical efficiency of 1064 nm laser from 2˜3% to over 50% without the need for a water-cooling system. The required power supply for the semiconductor laser has also been significantly reduced. Wherein, the power supply voltage of 1064 nm is within 48V, and the driving current is below 30A. Such low voltage and high current conditions can be directly generated by polymer lithium battery packs' direct discharge with high discharge rates, without the need for large volume capacitors. This facilitates the implementation of portable designs. Similarly, the second laser chip 42 and the third laser chip 43 are low-power semiconductor lasers within a power of 500 mW at wavelengths of 405 nm and 632 nm, respectively. Wherein, 405 nm is ultraviolet laser, which is mainly used for disinfection and sterilization of bacteria and viruses, while 632 nm red light utilizes photobiological effects to promote tissue repair. This design is also beneficial for achieving portable designs.

As shown in FIG. 2B, in this embodiment, the first laser chip 41 is an Edge Emitting semiconductor Laser (EEL), whose light emitting direction is aligned with the centerline of the output fiber optic 6, so that the emitted light of the first laser chip 41 is coupled to the output fiber optic 6 through the coupler 46. The first laser chip 41 is fixed inside an enclosed metal housing 40 through a heat sink 3. The housing 40 is connected to the air-cooled heat sink 5 to dissipate heat from the housing 40. Therefore, the air-cooled heat sink 5 can cool the first laser chip 41 through the housing 40 and heat sink 3 to reduce temperature drift and avoid burning the operator.

The second laser chip 42 is an edge emitting semiconductor laser, whose light emitting direction is perpendicular to the light emitting direction of the first laser chip 41. Besides, the second laser chip 42 is fixed to the housing 40 by means of the heat sink 3, also making use of the heat sink 3 and the housing 40, and cooled by the air-cooled heat sink 5.

Similarly, the third laser chip 43 is also an edge emitting semiconductor laser, whose light emitting direction is perpendicular to the light emitting direction of the first laser chip 41, and is fixed to the housing 40 by means of the heat sink 3 to be cooled by the air-cooled heat sink 5. However, compared with the second laser chip 42, the third laser chip 43 is closer to the coupler 46.

In other words, the second laser chip 42 and the third laser chip are both located between the first laser chip 41 and the coupler 46, wherein the light emitting direction of the first laser chip 41 passes through the optical center of the coupler 46 and is aligned with the centerline of the output fiber optic 6. The light emitting direction of the second laser chip 42 and the third laser chip is perpendicular to the light emitting direction of the first laser chip 41, and the distance from the third laser chip 43 to the coupler 46 is closer as compared with the second laser chip 42.

The optical center of the first optical mirror 44 is located in the light emitting direction of the first laser chip 41, and the first optical mirror 44 is tilted relative to the light emitting direction of the first laser chip 41, forming an angle. The incident surface of first optical mirror 44 towards the first laser chip 41 is coated with an antireflective film to theoretically transmit 100% of the emitted light of the first laser chip 41 (with a first wavelength, 1064 nm in this embodiment); the exit surface of the first optical mirror 44 towards the coupler 46 is coated with a reflective film to theoretically reflect 100% of the emitted light of the second laser chip 42 (with a second wavelength, 405 nm in this embodiment).

The optical center of the second optical mirror 45 is located in the light emitting direction of the first laser chip 41, and the second optical mirror 45 is tilted relative to the light emitting direction of the first laser chip 41, forming an angle. The incident surface of the second optical mirror 45 towards the first laser chip 41 is coated with an antireflective film to theoretically transmit 100% of the light beam (with a first wavelength) from the first laser chip 41 and the light beam (with a second wavelength different from the first wavelength) from the second laser chip 42. The exit surface of the second optical mirror 45 towards the coupler 46 is coated with a reflective film to theoretically reflect 100% of the light beam (with a third wavelength different from the first and second wavelengths) from the third laser chip 43.

The first optical mirror 44 and the second optical mirror 45 jointly achieve the selection of light beams of different wavelengths to enter the coupling mirror 46.

To be specific, in order to achieve disinfection and sterilization, the drive control circuit 1 sends out a first signal, so that the first laser chip 41 stops emitting light, the second laser chip 42 emits light, and the third laser chip stops emitting light. At the time, the emitted light of the second laser chip 42 is reflected by the first optical mirror 44 and is incident on the second optical mirror 45. Through the transmission of the second optical mirror 45, it is incident on the coupler 46 and coupled into the output fiber optic 6. In the end, it is incident on the surface of the human body through the hand tool 7 to disinfect and sterilize onychomycosis.

In order to implement treatment, the drive control circuit 1 sends out a second signal, the first laser chip 41 emits light, the second laser chip 42 and the third laser chip stop emitting light. At the time, the emitted light beam (1046 nm) of the first laser chip 41 through the transmission of the first optical mirror 44 and the second optical mirror 45 enters the coupler 46 and is coupled to the output fiber optic 6. At the point, the light beam of a first wavelength for treatment enters the hand tool 7 through the output fiber optic 6, then is incident on the surface of the human body for treatment.

In the end, to achieve accelerated rehabilitation, the drive control circuit 1 sends out a third signal, the first laser chip 41 and the second laser chip 42 both stop emitting light, while the third laser chip 43 emits light. At the time, the emitted light of the third laser chip 43 is reflected by the second optical mirror 45 and is incident on the coupler 46, then is incident the surface of the human body through the output fiber optic 6 and the hand tool 7.

In this way, three beams of laser of different wavelengths are combined through optical combiner lenses (first and second optical mirrors) coated with reflection/projection of different wavelengths, then are coupled to the output fiber optic through a coupling lens (coupler).

The hand tool 7 includes a moving piece 81, a convex lens 82, and a bracket 83. The bracket 83 serves as the light emitting end face for fitting to the skin, and has an opening in the light emitting direction of the output fiber optic to allow the emitted light of the output fiber optic to pass through the convex lens 82 and then emit from the opening to the skin.

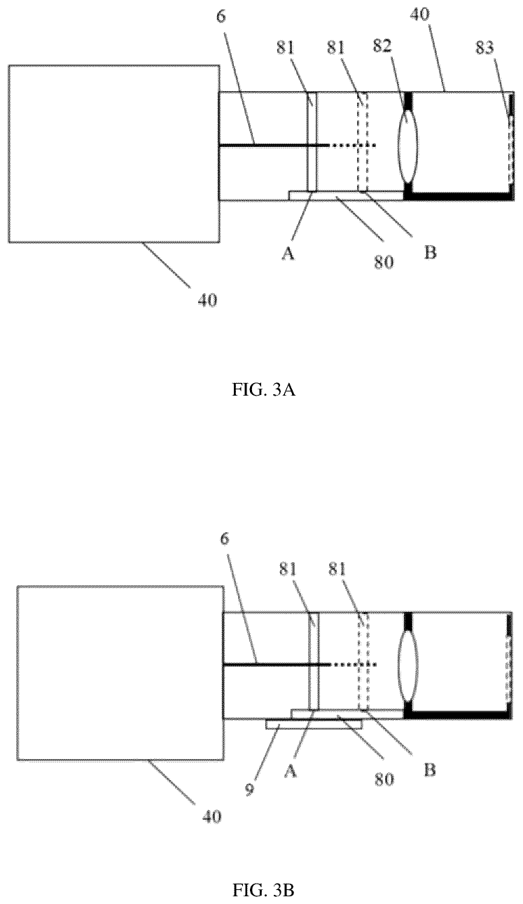

As shown in FIG. 3A, the light receiving end of the output fiber optic 6 is fixed inside the housing 40 and coupled with the coupler 46; the light emitting end is fixed on the moving piece 81. The moving piece 81 is fixed on the inner wall of the housing 40 through a slide rail 80. There are such two locators (e.g., blocks or slots) on the slide rail 80 as a first locator A and a second locator B to locate the moving piece 81 to the position shown in A or B in FIG. 3A. In other words, the moving piece 81 can move between the converging position (position A in the figure) and the diverging position (position B in the figure). It can be understood that two locators are not indispensable. Other mechanical structures can be employed to achieve the same function. All that is needed is to ensure that the moving piece 81 can switch between the converging position and the diverging position, and can be reliably located in the two positions without any deviation or movement.

When the moving piece 81 is in the converging position, the distance L from the light emitting end of the output fiber optic 6 to the optical center of the convex lens 82 satisfies: f<L<2f, wherein f represents the focal length of the convex lens. Therefore, the light beam emitted from the light emitting end of the output fiber optic 6 can converge into a small spot through the convex lens 82 and irradiate on the onychomycosis through an opening of the bracket 83. In one embodiment of the present disclosure, the diameter of the small spot is 2˜4 mm.

When the moving film 81 is in the diverging position, the distance L from the light emitting end of the output fiber optic 6 to the optical center of the convex lens 82 satisfies L<f. Therefore, the light beam emitted from the light emitting end of the output fiber optic 6 can diverge into a large spot through the convex lens 82 and irradiate on the onychomycosis through the opening on the bracket 83. In one embodiment of the present disclosure, the diameter of the large spot is 20˜100 mm, preferably 30˜50 mm.

The design of the above two types of light spots is aimed at improving the use effects of light beams of different wavelength and avoiding the negative effects of excessive laser irradiation on the human body surface. To be specific, laser of 405 nm wavelength is used for disinfection and sterilization, or laser of 632 nm wavelength is used for accelerated rehabilitation, which is generally continuous laser with power below several hundred milliwatts. If laser of 1064 nm wavelength is used for treatment, then the power of hundreds of watts of high energy pulse laser is far greater than the laser disinfection and sterilization of 405 nm wavelength, or the power of laser of 632 nm wavelength. For this reason, in the existing management system of medical devices, devices that adopt laser of 1064 nm wavelength for treatment are used in the department of dermatology; devices that adopt laser of 632 nm wavelength for accelerated rehabilitation are used in the department of physiotherapy. Because low-power and high-power instruments in medical devices have different indications, they need to be classified and managed. When approving medical devices, two different indications require two approval processes, which may lead to prolonged product launch times. Therefore, manufacturers are also divided into two independent entities. In addition, high-power laser instruments can only be operated by professionally trained personnel. These factors make it impossible for high-power (1064 nm wavelength) and low-power (405 nm wavelength and 632 nm wavelength) laser devices to be integrated into one device.

The laser disinfection and sterilization range of 405 nm and 632 nm wavelengths is large, requiring to be irradiated to a surface larger than the area of onychomycosis (large light spot is required) to avoid postoperative tissue infection caused by bacteria in the tissues around onychomycosis. The peak power required for laser therapy with a wavelength of 1064 nm is high, so it cannot be irradiated beyond the area of onychomycosis. Therefore, it must be a small light spot, and be smaller than the nail area. The commonly used light spot has a diameter of 2˜4 mm.

On the light emitting side of the output fiber optic 6, a convex lens 82 is installed inside the housing 40, with its optical center located in the light emitting direction of the output fiber optic, for converging or diverging the light emission of the fiber, thereby achieving the adjustment of large and small light spots.

On the light emitting side of the convex lens 82, there is an opening on the surface of the bracket 83, allowing the light beam to be emitted beyond the housing 40 to irradiate the surface of the onychomycosis and its surroundings. The bracket 83 serves as the light emitting end face of the housing 40, for close contact with the surface of the skin, so that the distance between the skin surface and the optical center of the convex lens 82 maintains a constant working distance (image distance). The diameter of the large or small spot is based on the spot diameter at bracket 83.

Due to the extremely high electro-optical efficiency of semiconductor lasers, the system generates very little heat. Therefore, semiconductor lasers only require simple air-cooled heat sinks to complete heat dissipation, resulting in a significant reduction in volume and weight. In addition, semiconductor lasers use the cleavage surface of a wafer as the laser resonant cavity, which is self-contained and does not require adjustment. It is simple, reliable, compact, and insensitive to impact vibration, making it easy to integrate in portable devices.

### Second Embodiment

The multi-wavelength laser treatment device for trichophyton rubrum induced onychomycosis provided in this embodiment differs from the first embodiment in that the method of controlling the position switching of the moving piece is different, while the rest are the same.

In the first embodiment, the drive control circuit 1 is used to control the position switching of the moving piece 81, that is, circuit control. In this embodiment, the slide key 9 outside the housing 40 is used to push the moving piece 81, thereby achieving mechanical control.

### Third Embodiment

In the third embodiment of the present disclosure, a method of using the multi-wavelength laser treatment device of the first embodiment is further provided, comprising the following steps:
S1: Use drive control circuit 1 to move the light emitting end of an output fiber optic to a diverging position (B).

In this embodiment, circuit control is used to drive the moving piece 81 to move to the diverging position (B), namely, move the light emitting end of the output fiber optic 6 to the diverging position.
S2: Use drive control circuit 1 to power on one of the three chips to emit a light beam of 405 nm wavelength, couple to the output fiber optic and then diverge into a large spot through a convex lens for output.

In this embodiment, a light beam of 405 nm wavelength is emitted from the second laser chip 42, which is reflected by the first optical mirror 44 and incident onto the second optical mirror 45. Then, through the transmission of the second optical mirror 45, it is coupled to the output fiber optic 6 by the coupler 46. Furthermore, through the light emitting end of the fiber 6 (in the diverging position at the time), it is incident onto the convex lens 82. Since the light emitting end is in the diverging position at the time, that is, within the focal length f of the convex lens 82, the light beam emitted from the output fiber optic 6 is diverged and amplified into a large light spot by the convex lens 82, to disinfect and sterilize the onychomycosis surface and its surroundings. At the time, irradiation is at low power.
S3: After a first preset time, stop the light beam of 405 nm wavelength and move the light emitting end of the output fiber optic to a converging position.

In this embodiment, the onychomycosis surface and its surroundings are disinfected and sterilized with a light beam of 405 nm wavelength and a large spot of 30 mm, and the irradiation time is 10˜60 seconds (the first preset time). Then, use the drive control circuit 1 to move the moving piece 82 to the converging position.
S4: Use drive control circuit 1 to power on one of the three chips to emit a light beam of 1064 nm wavelength, couple to the output fiber optic and then converge into a small spot through a convex lens for output.

Similarly, use the drive control circuit 1 to make the first laser chip 41 emit a light beam of 1064 nm wavelength, which is transmitted through the first optical mirror 44 and incident onto the second optical mirror 45. Then, through the transmission of the second optical mirror 45, it is coupled to the output fiber optic 6 through the coupler 46. Further, through the light emitting end (in the converging position at the time) of the output fiber optic 6, it is incident onto the convex lens 82. Because the light emitting end is in the converging position at the time, the light beam emitted from the output fiber optic 6 is converged into a small spot by the convex lens 82, and only the surface of onychomycosis is irradiated for treatment.

Moreover, it is necessary to use the drive control circuit 1 to increase the current of the first laser chip (this current is greater than the current provided to the second laser chip), in order to increase the energy of the light beam of 1064 nm wavelength to several hundred watts required for treatment (such as 300˜500W).
S5: After a second preset time, stop the light beam of 1064 nm wavelength and move the light emitting end of the output fiber optic to a diverging position.

In this embodiment, a light beam of 1064 nm wavelength and a small spot with a diameter of 3 mm are used to irradiate the surface of onychomycosis. The irradiation time is determined by the operator based on the condition of the disease, for example, the irradiation time is between 5˜30 minutes (the second preset time). Then, use the drive control circuit 1 to move the moving piece 82 to the divergent position.
S6: Use drive control circuit 1 to power on one of the three chips to emit a light beam of 632 nm wavelength, couple to the output fiber optic and then diverge into a large spot through a convex lens for output.

In this embodiment, the third laser chip 43 emits a light beam of 632 nm wavelength, which is reflected by the second optical mirror 45 and coupled to the output fiber optic 6 by the coupler 46. Then, through the light emitting end of fiber 6 (in the diverging position at the time), it is incident onto the convex lens 82. Because the light emitting end is in the divergent position at the time, the light beam emitted from the output fiber optic 6 is diverged and amplified into a large spot by the convex lens 82, which irradiates the onychomycosis surface and its surroundings.
S7: After a third preset time, stop the light beam of 632 nm wavelength and end the process.

In this embodiment, a light beam of 632 nm wavelength and a large spot with a diameter of 30 mm are used to irradiate the onychomycosis surface and its surroundings, with an irradiation time of 10˜30 minutes (the third preset time). At the time, the drive control circuit 1 provides a current to the third laser chip that is smaller than the current provided to the first laser chip (i.e., the current provided to the first laser chip is the largest, so that it has high power and high energy for treatment), for irradiation in a low-power state.

To sum up, the multi-wavelength laser treatment device for trichophyton rubrum induced onychomycosis provided in the embodiments of the present disclosure integrates three wavelengths, and for the harsh sanitary conditions in the field, completes the process of disinfection and sterilization (405 nm), treatment (1064 nm) and rehabilitation (632 nm) at one time, thereby reducing the number of outpatient visits. Moreover, the use of high electro-optical efficiency semiconductor lasers instead of solid-state lasers has achieved air-cooled design to reduce energy consumption and realized portable design. In addition, two designs are adopted: lithium batteries and charging circuits, to adapt to various usage environments and increase battery endurance.

It should be noted that the above multiple embodiments are only examples, and the technical solutions of the various embodiments can be combined, all of which are within the scope of protection of the present disclosure. The sequence of steps in the present disclosure can be changed according to actual needs, and the order of steps can be altered. Serial processing can also be changed to parallel processing, not limited to the sequence of steps listed in the embodiments.

Besides, the terms “first” and “second” are only used for the purpose of description and shall not be understood as indicating or implying relative importance or implying the quantity of technical features indicated. Therefore, the features limited to “first” and “second” can explicitly or implicitly include one or more of these features. In the description of the present disclosure, the meaning of “more” is two or more than two, unless otherwise specified.

The above is a detailed explanation of the multi-wavelength laser treatment device and usage method provided by the present disclosure for trichophyton rubrum induced onychomycosis. For a person having ordinary skill in the art, any obvious modifications made to the present disclosure without deviating from the substantive content of the present disclosure will constitute infringement of the patent rights of the present disclosure and will bear corresponding legal responsibilities.

1. A multi-wavelength laser treatment device for trichophyton rubrum induced onychomycosis, comprising: a drive control circuit, a power supply, an optical system, an air-cooled heat sink, an output fiber optic, and a hand tool; wherein,
the hand tool includes a moving piece, a convex lens, and a bracket, and the bracket serves as a light emitting end face for fitting to the skin,
the drive control circuit is powered by the power supply and controls the optical system, air-cooled heat sink, moving piece, and convex lens,
the optical system includes a first laser chip, a second laser chip, a third laser chip, a first optical mirror, a second optical mirror, and a coupler,
a light beam is coupled to the output fiber optic through the coupler and emits light from the light emitting end face of the hand tool; wherein, the first laser chip, the second laser chip and the third laser chip have a light emitting wavelength of 405 nm, 1064 nm or 632 nm respectively, for emitting light beams of 405 nm, 1064 nm and 632 nm wavelengths in turn; the 405 nm is used for disinfection and sterilization of bacteria and viruses attached to a surface of and around the onychomycosis, the 1064 nm is used to treat the trichophyton rubrum induced onychomycosis, and the light beam of 632 nm wavelength is used for rehabilitation;
the moving piece is used to fix a light emitting end of the output fiber optic and can switch between a converging position and a diverging position; when the moving piece is in the converging position, a distance L from the light emitting end of the output fiber optic to an optical center of the convex lens satisfies: f<L<2f; when the moving piece is in the diverging position, the distance L from the light emitting end of the output fiber optic to the optical center of the convex lens satisfies: L<f.

2. The multi-wavelength laser treatment device of claim 1, wherein: the second laser chip and the third laser chip are both located between the first laser chip and the coupler,
a light emitting direction of the first laser chip passes through an optical center of the coupler and is aligned with a centerline of the output fiber optic,
a light emitting direction of the second laser chip and the third laser chip is perpendicular to the light emitting direction of the first laser chip, and the third laser chip is closer to the coupler compared with the second laser chip.

3. The multi-wavelength laser treatment device of claim 2, wherein:
the optical center of the first optical mirror is located in the light emitting direction of the first laser chip, and the first optical mirror is tilted relative to the light emitting direction of the first laser chip.

4. The multi-wavelength laser treatment device of claim 3, wherein:
an incident surface of the first optical mirror towards the first laser chip is coated with an antireflective film to transmit emitted light of the first laser chip;
an exit surface of the first optical mirror towards the coupler is coated with a reflective film to reflect emitted light of the second laser chip.

5. The multi-wavelength laser treatment device of claim 4, wherein:
the optical center of the second optical mirror is located in the light emitting direction of the first laser chip, and the second optical mirror is tilted relative to the light emitting direction of the first laser chip.

6. The multi-wavelength laser treatment device of claim 5, wherein:
an incident surface of the second optical mirror towards the first laser chip is coated with an antireflective film to transmit a light beam from the first laser chip and a light beam from the second laser chip;
an exit surface of the second optical mirror towards the coupler is coated with a reflective film to reflect a light beam from the third laser chip.

7. A method of using the multi-wavelength laser treatment device according to claim 1, comprising the following steps to:
S1: use the drive control circuit to move the light emitting end of the output fiber optic to the diverging position;
S2: use the drive control circuit to power on one of the first laser chip, second laser chip, or third laser chip to emit a light beam of 405 nm wavelength, couple it to the output fiber optic, and then diverge into a large spot through the convex lens for output;
S3: after a first preset time, stop the light beam of 405 nm wavelength and move the light emitting end of the output fiber optic to the converging position;
S4: use the drive control circuit to power on one of the first laser chip, second laser chip, or third laser chip to emit a light beam of 1064 nm wavelength, couple it to the output fiber optic, and then converge into a small spot through the convex lens for output;
S5: after a second preset time, stop the light beam of 1064 nm wavelength and move the light emitting end of the output fiber optic to the diverging position;
S6: use the drive control circuit to power on one of the first laser chip, second laser chip, or third laser chip to emit a light beam of 632 nm wavelength, couple it to the output fiber optic, and then diverge into a large spot through the convex lens for output, a diameter of the large spot being larger than that of the small spot;
S7: after a third preset time, stop the light beam of 632 nm wavelength and end.

8. The method of using the multi-wavelength laser treatment device according to claim 7, wherein:
a current provided by the drive control circuit to the first laser chip is greater than a current provided to the second and third laser chips.

9. The method of using the multi-wavelength laser treatment device according to claim 8, wherein:
the first laser chip is used to provide the light beam of 1064 nm wavelength; the second laser chip is used to provide the light beam of 405 nm wavelength; and the third laser chip is used to provide the light beam 632 nm wavelength.

10. The method of using the multi-wavelength laser treatment device according to claim 9, wherein:
the first preset time is 10˜60 seconds, and the third preset time is 10˜30 minutes.

11. The method of claim 7, wherein:
the second laser chip and the third laser chip are both located between the first laser chip and the coupler,
a light emitting direction of the first laser chip passes through an optical center of the coupler and is aligned with a centerline of the output fiber optic,
a light emitting direction of the second laser chip and the third laser chip is perpendicular to the light emitting direction of the first laser chip, and the third laser chip is closer to the coupler compared with the second laser chip.

12. The method of claim 11, wherein:
the optical center of the first optical mirror is located in the light emitting direction of the first laser chip, and the first optical mirror is tilted relative to the light emitting direction of the first laser chip.

13. The method of claim 12, wherein:
an incident surface of the first optical mirror towards the first laser chip is coated with an antireflective film to transmit emitted light of the first laser chip;
an exit surface of the first optical mirror towards the coupler is coated with a reflective film to reflect emitted light of the second laser chip.

14. The method of claim 13, wherein:
the optical center of the second optical mirror is located in the light emitting direction of the first laser chip, and the second optical mirror is tilted relative to the light emitting direction of the first laser chip.

15. The method of claim 14, wherein:
an incident surface of the second optical mirror towards the first laser chip is coated with an antireflective film to transmit a light beam from the first laser chip and a light beam from the second laser chip;
an exit surface of the second optical mirror towards the coupler is coated with a reflective film to reflect a light beam from the third laser chip.
